(19) United States
(12) Patent Application Publication
(10) Pub. No.: US 2006/0247248 A1
(43) Pub. Date: Nov. 2, 2006

(54) Sigma-1 receptor ligand with acetylcholinesterase

(76) Inventors: Vassilios Papadopoulos, North Potomac, MD (US); Laurent Lecanu, McLean, VA (US); Janet Greeson, Las Vegas, NV (US)

Correspondence Address: Schwegman, Lundberg, Woessner & Kluth, P.A., P.O. Box 2938, Minneapolis, MN, 55402, US

(21) Appl. No.: 11/347,020

(22) Filed: Feb. 3, 2006

Related U.S. Application Data
(63) Continuation-in-part of application No. PCT/US04/25295, filed on Aug. 5, 2004.
(60) Provisional application No. 60/492,769, filed on Aug. 5, 2003.

(51) Int. Cl. A61K 31/495; C07D 241/04

(52) U.S. Cl. 514/252.12; 544/400

(57) ABSTRACT

A novel compound and method for preventing or treating neurodegenerative diseases by inhibiting acetylcholinesterase and binding the sigma-1 receptor are disclosed. Dimethylcarbamic acid 2,3-bis-dimethylcarbamoyloxy-6-[4-(4-ethyl-piperazin-yl)-butyryl]-phenyl ester and its derivatives represent a novel therapeutic strategy against β-amyloid peptide induced neurotoxicity, in inhibiting acetylcholinesterase, in improving cholinergic transmission, in binding the sigma-1 receptor, and in releasing a metabolite that is active both as a sigma-1 receptor ligand and an antioxidant.

### REFERENCE TO RELATED APPLICATIONS

This application is a continuation in part and a U.S. National Stage Application of PCT/US04/025295 filed Aug. 5, 2004 and published in English as WO 05/016276 on Feb. 24, 2005 which claims priority under 35 U.S.C. 119(e) from U.S. Provisional Application Ser. No. 60/492,769 filed Aug. 5, 2003, which applications and publication are herein incorporated by reference.

### FIELD OF THE INVENTION

The present invention relates generally to a method of preventing or treating a neurodegenerative disease by inhibiting acetylcholinesterase, and particularly to therapeutic compounds and pharmaceutical compositions for preventing or treating neurodegenerative diseases or disorders that involve nerve cell death.

### BACKGROUND OF THE INVENTION

Nerve cell degeneration and death can cause potentially devastating and irreversible effects in an individual and may occur as a result of stroke, heart attack, exposure to a neurotoxic agent, such as nerve gas, or other brain or spinal chord ischemia or trauma, among other things. Neurodegenerative disorders that involve nerve cell death include Alzheimer's disease, Parkinson s disease, Huntington s disease, Amyotrophic Lateral Sclerosis, Down's Syndrome, and Korsakoff s disease.

Alzheimer s disease (AD) is a degenerative disease of nerve cells in the cerebral cortex that leads to atrophy of the brain and senile dementia. AD currently affects more than four million people in the United States and is the most common form of dementia in the elderly, affecting about 10% of people over the age of 65 and about 40% of people over 80. Familial AD is the early-onset form of the disease involving mutations of the amyloid protein precursor (APP) gene and accounts for about 5% of the total AD cases. Sporadic AD is the late-onset form of the disease and accounts for about 95% of the total AD cases. The cause of sporadic AD remains unknown. The average duration of AD is eight years. AD is a costly disease, in terms of medical care, nursing home care, social services, lost productivity, and shortened life span.

Clinically, AD is characterized by a progressive and irreversible impairment of cognitive processes and memory alteration. Histologically, AD is characterized by the presence in the brain of neuritic plaques from deposition of insoluble amyloid aggregates, the formation of neurofibrillay tangles from hyperphosphorylation of the tau protein, and the degeneration of cholinergic neurons.

Current therapeutic strategies for AD include inhibitors of β-amyloid peptide (Aβ) production, compounds that prevent Aβ oligomerization and fibrillization, anti-inflammatory agents, inhibitors of cholesterol synthesis, antioxidants, neurorestorative agents, and vaccines. Selkoe, Nature, 399:A23-31 (1999); Emilien et al., Arch. Neurol., 57:454-459 (2000); Klein, Neurochem. Int., 41:345-352 (2002); Helmuth, Science, 297:1260-1262 (2002). The scientific community has focused mainly on improving the cholinergic network dysfunction and created a class of acetylcholinesterase (AchE) inhibitors as therapeutics. However, despite promising clinical data, the beneficial effects of a leading AchE inhibitor, tacrine, were modest.

In addition, a new generation of AchE inhibitors, represented by galantamine and donezepil, did not further delay the onset of symptoms. Targeting AchE solely has proven to be limiting, as the currently known AchE inhibitors delay the onset of symptoms for only one to two years, during which the cholinergic neurons progressively degenerate. Tariot & Winblad, Alzheimer's disease: advances in etiology, pathogenesis and therapeutics, 707-723 (2001) (ed. Iqbal et al.); Waldemar et al., Alzheimer s disease: advances in etiology, pathogenesis and therapeutics, 725-738 (2001) (ed. Iqbal et al.). With the exception of memantine, an antagonist of the glutamatergic NMDA-subtype receptor, no further advances have been made in Alzheimer's disease therapeutics.

Recent experimental data suggest, however, that the sigma-1 (σ-1) receptor would be an attractive target for developing candidates for AD therapeutics. The σ-1 receptor is present in different brain structures, such as the cortex or the hippocampus, localized on cell membranes, endoplasmic reticulum membranes, and mitochondrial membranes. Alonso et al., Neuroscience, 97:155-170 (2000). The significance of the different sub-cellular localizations of the σ-1 receptor remains unknown. However, σ-1 receptor agonists are known to protect neuronal cells against cerebral ischemia in rat, to exert antidepressant effects in Aβ₂₅₋₃₅ treated mice, to enhance the acetylcholine release in the rat brain, and to facilitate neurite sprouting in PC12 cells induced by nerve growth factor. Kume et al., Eur. J. Pharmacol., 455:91-100 (2002); Goyagi et al., Anesth. Analg., 96:532-538 (2003); Urani et al., Behav. Brain Res., 134:239-247 (2002); Kobayashi et al., J. Pharmacol. Exp. Ther., 279:106-113 (1996); Takebayashi et al., J. Pharmacol. Exp. Ther., 303:1227-1237 (2002).

Several σ-1 receptor agonists have been described to reverse, in a dose-dependent manner, scopolamine-induced amnesia in rats. Maurice et al., Brain Res. Rev., 37:116-132 (2001). SA4503, a σ-1 receptor agonist, enhanced the acetylcholine release in the hippocampus of rat brain slices and in vivo, suggesting that the anti-amnesic effect could be due in part to the activation of the cholinergic pathway. Horan et al., Synapse, 46:1-3 (2002); Kobayashi et al., J. Pharmacol. Exp. Ther., 279:106-113 (1996). The effect of SA4503 on the release of acetylcholine appeared to be more pronounced than with tacrine. Kobayashi et al., J. Pharmacol. Exp. Ther., 279:106-113 (1996). Igmesine, another σ-1 receptor agonist, has been recently demonstrated to exert antidepressant activity on mice that were intracerebroventrically injected with the amyloid fragment Aβ₂₅₋₃₅, probably by a modification of the monoaminergic system. Urani et al., Behav. Brain Res., 134:239-247 (2002); Akunne et al., Neuropharmacology, 41:138-149 (2001). This antidepressant effect was observed with yet another σ-1 receptor agonist, PRE-084, in mice submitted to the forced swimming test. Urani et al., J. Pharmacol. Exp. Ther., 298:1269-1279 (2001).

The antidepressant activity displayed by σ-1 receptor agonists involves a modulation of intracellular calcium mobilization, partly through regulation of the ryanodine receptor. Urani et al., Psychopharmacology, 163:26-34 (2002); Hayashi et al., J. Pharmacol. Exp. Ther. 293:788-798 (2000). The disruption of calcium homeostasis, which leads to a pathological alteration of calcium signaling, is currently a theory proposed to explain the origin of AD. Kachaturian, Neurobiol. Aging, 8:345-346 (1987). In fact, much data has been published that highlight the role of calcium in the pathogenesis of AD, but the use of different calcium inhibitors to slow down the progression of AD and to reverse the memory alteration remains unsuccessful.

Therapeutics for the prevention or treatment of AD thus remains relatively unsuccessful. New compounds or agents for therapeutic regimes to inhibit AchE, so as to improve the cholinergic network dysfunction, and to bind the σ-1 receptor, so as to protect neuronal cells and exert beneficial effects in neurodenegerative diseases or disorders, would be valuable.

### SUMMARY OF THE INVENTION

The invention provides compounds, pharmaceutical compositions and methods for preventing or treating neurodegenerative diseases and disorders by inhibiting acetylcholinesterase and binding the sigma-1 receptor. Thus, the present invention provides novel compounds of formula I:

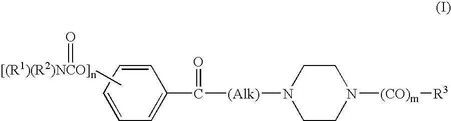

a) R¹ and R² are individually H, (C₁-C₆)alkyl, (C₃-C₆)cycloalkyl, (C₃-C₆)cycloalkyl((C₁-C₆)alkyl), (C₂-C₆)alkenyl, wherein cycloalkyl optionally comprises 1-2, S, nonperoxide O or N(R¹); aryl, aryl(C₁-C₆)alkyl, aryl(C₂-C₆)alkenyl, heteroaryl, heteroaryl(C₁-C₆)alkyl, or R¹ and R² together with the N to which they are attached form a 5- or 6-membered heterocyclic or heteroaryl ring, optionally substituted with R¹ and optionally comprising 1-2, S, non-peroxide O or N(R¹);

b) (Alk) is (C₁-C₆)alkyl, (C₂-C₆)alkenyl, (C₃-C₆)cycloalkyl, (C₃-C₆)cycloalkyl(C₂-C₆)alkyl or [(C₂-C₆)alkyl(C₃-C₆)cycloalkyl[(C₃-C₆)alkyl], each optionally substituted by 1-2 S, non-peroxide O or N(R¹);

c) n is 1, 2 or 3;

d) m is 0 or 1;

e) R³ is H, OH, (C₁-C₆)alkyl, (C₁-C₆)alkoxy, (C₃-C₆)cycloalkyl, (C₃-C₆)cycloalkyl((C₁-C₆)alkyl), (C₂-C₆)alkenyl, (C₂-C₆)alkynyl, (C₁-C₆)alkanoyl, halo(C₁-C₆)alkyl, hydroxy(C₁-C₆)alkyl, (C₁-C₆)alkoxycarbonyl; (C₁-C₆)alkylthio, thio(C₁-C₆)alkyl or (C₁-C₆)alkanoyloxy; or a pharmaceutically-acceptable salt thereof.

Preferably n is 2 or 3, most preferably 3, wherein the carbamoyl substituents are preferably in the 2, 3 and 4 positions on the phenyl ring. Preferably, R¹, R² and R³ are individually (C₁-C₆)alkyl, (C₃-C₆)cycloalkyl or (C₃-C₆)cycloalkyl(C₁-C₆)alkyl; most preferably (C₁-C₄)alkyl, such as methyl, ethyl, butyl, or propyl. Preferably (Alk) is (C₁-C₆)alkyl, most preferably —(CH₂)₁₋₄—, such as —(CH₂)₃—. Preferably m is 0 and R³ is ethyl. Piperazinyl may be optionally substituted with 1-2 methyl or ethyl groups. One or more of the moieties [(R¹)(R²)NC(O)O] may be replaced by [HO—] groups, to yield compounds of formula II that are expected to exhibit antioxidant properties. Some of the compounds of formula (I) are useful intermediates for the preparation of other compounds of formula (I).

The present invention also provides pharmaceutical compositions comprising an effective amount of a compound of formula (I) in combination with a pharmaceutically-acceptable carrier and/or excipient(s), as well as a method to use such compounds and compositions to treat a neurodegenerative or neuropathological condition by administration of an effective amount or dosage thereof to a mammal, such as a human, afflicted with, or threatened by the onset of such a condition.

Compounds of formula (I) can be readily prepared by reacting protected phenols of general formula (PO)ₙPh wherein P is a removable hydroxyl protecting group, n is 1-3 and Ph is a benzene or other aryl ring, with an acid chloride of general formula ClC(O)-(Alk)-Cl in the presence of AlCl₃. The resulting product can be reacted with a 1-substituted piperazine, with the 1-C(O)R³ group reduced and/or protected as necessary, followed by deprotection of the phenolic OH groups and acyl group, as needed, to yield a compound of general formula (II): (4 -substituted-piperazin-1-yl)(Alk)C(O)Ph(OH)ₙ (II) wherein n is 1-3, and piperazin-1-yl is 4-substituted with (C(O))ₘR³). Bioactive compounds of formula (II) are also within the scope of the invention. The phenolic OH groups are then carbamoylated using a compound of general formula (R¹)(R²)NC(O)Cl to yield compounds of formula (I). FIG. 2 depicts the preparation of a representative compound of the invention, which is shown in FIG. 1.

Compounds of formula (I), such as dimethyl-carbamic acid 2,3-bis-dimethylcarbamoyloxy-6-[4-(4-ethyl-piperazin-yl)-butyryl]-phenyl ester can be used to inhibit acetylcholinesterase and bind the sigma-1 receptor, and also to generate metabolites that are active as both sigma-1 receptor ligands and as antioxidants.

A therapeutically effective amount of the novel compound of the invention can be administered as a pharmaceutical composition to improve cholinergic transmission and prevent or reduce β-amyloid peptide induced neurotoxicity in the brain, and thereby prevent or treat neurodegenerative diseases and disorders. In one aspect, the invention provides a pharmaceutical composition that includes a therapeutically effective amount of a novel compound and a pharmaceutically acceptable carrier.

In another aspect, the invention provides a method for synthesizing the novel compound of the invention. In yet another aspect, the invention provides a method for preventing or treating a neurodegenerative disease or disorder in a subject by administering a therapeutically effective amount of the novel compound of the invention. In another aspect, novel compounds or intermediates depicted or disclosed herein are within the scope of the invention.

The details of one or more embodiments of the invention are set forth in the accompanying description below. Other features, objects, and advantages of the invention will be apparent from the description and claims.

### BRIEF DESCRIPTION OF THE DRAWINGS

FIG. 1 shows the chemical structure of dimethyl-carbamic acid 2,3-bis-dimethylcarbamoyloxy-6-[4-(4-ethyl-piperazin-yl)-butyryl]-phenyl ester (SP004);

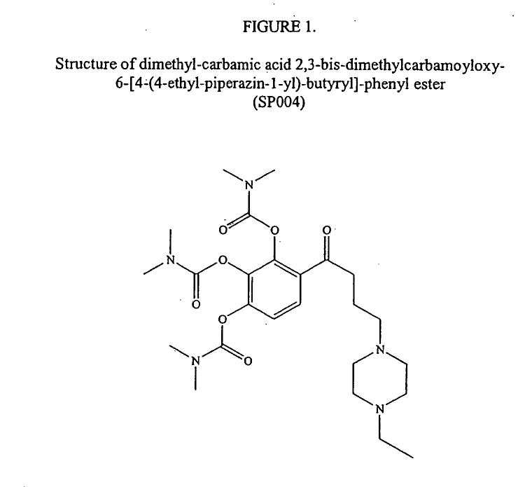

FIG. 2 is a flowchart of steps in SP004 synthesis;

FIG. 3 is a graph depicting a competition curve for specific binding of SP004 vs. haloperidol at the human σ-1 receptor;

FIG. 4 is a graph depicting a competition curve for specific binding of SP004 vs. haloperidol at the human σ-2 receptor;

FIG. 5 is a graph depicting the effects of SP004 on human AchE activity;

FIG. 6 shows the mechanism of inactivation of AchE by SP004 and production of an active metabolite; and

FIG. 7 shows the chemical structure of 4-(4-ethyl-piperazin-yl)-1-(2,3,4-trihydroxy-phenyl)-butan-1-one (SP004m).

### DETAILED DESCRIPTION

While the present invention may be embodied in many different forms, several specific embodiments are discussed herein with the understanding that the present disclosure is to be considered only as an exemplification of the principles of the invention, and it is not intended to limit the invention to the embodiments illustrated.

Abbreviations used are as follows: acetylcholine (Ach); acetylcholinesterase (AchE); acetylcholinesterase inhibitor (AchEI); Alzheimer's disease (AD); β-amyloid peptide (Aβ); dimethyl-carbamic acid 2,3-bis-dimethylcarbamoyloxy-6-[4-(4-ethyl-piperazin-yl)-butyryl]-phenyl ester (SP004); 4-(4-ethyl-piperazin-yl)-1-(2,3,4-trihydroxy-phenyl)-butan-1-one (SP004m); sigma-1 receptor (σ-1 receptor); sigma-2 receptor (σ-2 receptor).

The term “cortisol-modulating agent” means any agent possessing pharmacological activity as regulating, preventing or decreasing any pathological raise of cortisol synthesis, rebalancing, or tending to re-balance, cortisol synthesis, therefore the intensity of the cortisol effect at a physiological activity. The definition of “cortisol-modulating agent“ ” as used herein can also mean that the agent possessing cortisol-modulating or -regulating or -re-balancing pharmacological activity may, if desired, be in the form of a free base, a free acid, a salt, an ester, a hydrate, an amide, an enantiomer, an isomer, a tautomer, a prodrug, a polymorph, a derivative or the like, provided the free base, free acid, salt, ester, hydrate, amide, enantiomer, isomer, tautomer, prodrug, or derivative is suitable pharmacologically, that is, effective in the present methods, combinations, kits, and compositions.

The terms “therapeutically effective amount,” “effective amount,” or “pharmacologically effective amount” refer to the amount of the compound that is required to confer therapeutic effect on the treated subject.

The term “derivative” refers to a compound that is produced from another compound of similar structure by the replacement or substitution of one atom, molecule, or group by another.

The term “bioavailability” refers to the extent to which an active moiety (drug or metabolite) is absorbed into the general circulation and becomes available at the site of drug action in the body.

The term “combination therapy” embraces the administration of at least one composition of the present invention in conjunction with another pharmaceutical agent that is indicated for treating or preventing a neurodegenerative disease or disorder in a subject, as part of a specific treatment regimen intended to provide a beneficial effect from the co-action of these therapeutic agents for the treatment of a neurodegenerative disease or disorder.

The term “prevent” or “prevention” in relation to a neurodegenerative disease or disorder in a subject, means no disease or disorder development if none had occurred, or no further disorder or disease development if there had already been development of the disorder or disease.

The term “prodrug” refers to a drug or compound (active principal) that elicits the pharmacological action resulting from conversion by metabolic processes within the body. Prodrugs are generally considered drug precursors that, following administration to a subject and subsequent absorption, are converted to an active or a more active species via some process, such as a metabolic process. Other products from the conversion process are easily disposed of by the body. Prodrugs generally have a chemical group present on the prodrug which renders it less active and/or confers solubility or some other property to the drug. Once the chemical group has been cleaved from the prodrug the more active drug is generated. Prodrugs may be designed as reversible drug derivatives and utilized as modifiers to enhance drug transport to site-specific tissues. The design of prodrugs to date has been to increase the effective water solubility of the therapeutic compound for targeting to regions where water is the principal solvent. For example, Fedorak et al., Am. J. Physiol., 269:G210-218 (1995), describe dexamethasone-beta-D-glucuronide. McLoed et al., Gastroenterol., 106:405-413 (1994), describe dexamethasonesuccinate-dextrans. Hochhaus et al., Biomed. Chrom., 6:283-286 (1992), describe dexamethasone-21-sulphobenzoate sodium and dexamethasone-21-isonicotinate. Additionally, J. Larsen and H. Bundgaard, Int. J. Pharmaceutics, 37, 87 (1987) describe the evaluation of N-acylsulfonanlides as potential prodrug derivatives. J. Larsen et al., Int. J. Pharmaceutics, 47, 103 (1988) describe the evaluation of N-methylsulfonamides as potential prodrug derivatives. Prodrugs are also described in, for example, Sinkula et al., J. Pharm. Sci., 64:181-210 (1975).

The term “treat” or “treatment” as used herein refers to any treatment of a disorder or disease associated with a neurodegenerative disease or disorder or neuropathology, in a subject, and includes, but is not limited to, preventing the disorder or disease from occurring in a subject who may be predisposed to the disorder or disease, but has not yet been diagnosed as having the disorder or disease; inhibiting the disorder or disease, for example, arresting the development of the disorder or disease; relieving the disorder or disease, for example, causing regression of the disorder or disease; or relieving the condition caused by the disease or disorder, for example, stopping one or more symptoms of the disease or disorder.

The following definitions are used, unless otherwise described: halo is fluoro, chloro, bromo, or iodo, alkyl, alkoxy, alkenyl, alkynyl, etc. denote both straight and branched groups; but reference to an individual radical such as “propyl” embraces only the straight chain radical, a branched chain isomer such as isopropyl” being specifically referred to. Aryl denotes a phenyl radical or an orthofused bicyclic carbocyclic radical having about nine to ten ring atoms in which at least one ring is aromatic. Heteroaryl encompasses a radical attached via a ring carbon of a monocyclic aromatic ring containing about 5 or 6 ring atoms consisting of carbon and one to four heteroatoms each selected from the group consisting of non-peroxide oxygen, sulfur, and N(R¹) wherein R² is absent or is as defined above; as well as a radical of an ortho-fused bicyclic heterocycle of about eight to ten ring atoms derived therefrom, particularly a benz-derivative or one derived by fusing a propylene, trimethylene, or tetramethylene diradical thereto.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active polymorphic, or stereoisomeric form, or mixtures thereof, of a compound of the invention, which possess the useful properties described herein, it being well known in the art how to prepare optically active forms (for example, by resolution of the racemic fom by recrystallization techniques, by synthesis from optically-active starting materials, by chiral synthesis, or by chromatographic separation using a chiral stationary phase) and how to determine anti-toxin activity using the standard tests described herein, or using other similar tests which are well known in the art.

Specific and preferred values listed below for radicals, substituents, and ranges, are for illustration only; they do not exclude other defined values or other values within defined ranges for the radicals and substituents.

Specifically, (C₁-C₆)alkyl can be methyl, ethyl, propyl, isopropyl, butyl, isobutyl, sec-butyl, pentyl, 3-pentyl, or hexyl; (C₃-C₁₂)cycloalkyl can be monocyclic bicyclic or tricyclic and includes cyclopropyl, cyclobutyl, cyclopentyl, cyclohexyl, bicycle[2.2.2]octanyl, norbornyl, adamantyl as well as various terpene and terpenoid structures. (C₃-C₁₂)cycloalkyl(C₁-C₆)alkyl includes the foregoing cycloalkyl and can be cyclopropylmethyl, cyclobutylmethyl, cyclopentylmethyl, cyclohexylmethyl, 2-cyclopropylethyl, 2-cyclobutylethyl, 2-cyclopentylethyl, or 2-cyclohexylethyl. Heterocycloalkyl and (heterocycloalkyl)alkyl include the foregoing cycloalkyl wherein the cycloalkyl ring system is monocyclic, bicyclic or tricyclic and optionally comprises 1-2 S, non-peroxide 0 or N(R⁷) as well as 2-12 ring carbon atoms; such as morpholinyl, piperidinyl, piperazinyl, indanyl, 1,3-dithian-2-yl, and the like; The cycloalkyl ring system optionally includes 1-3 double bonds or epoxy moieties and optionally is substituted with 1-3 OH, (C₁-C₆)alkanoyloxy, (CO), (C₁-C₆)alkyl or (C₂-C₆)alkynyl. (C₁-C₆)alkoxy can be methoxy, ethoxy, propoxy, isopropoxy, butoxy, iso-butoxy, sec-butoxy, pentoxy, 3-pentoxy, or hexyloxy; (C₂-C₆)alkenyl can be vinyl, allyl, 1-propenyl, 2-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, or 5-hexenyl; (C₂-C₆)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl; (C₁-C₆)alkanoyl can be formyl, acetyl, propanoyl or butanoyl; halo(C₁-C₆)alkyl can be iodomethyl, bromomethyl, chloromethyl, fluoromethyl, trifluoromethyl, 2-chloroethyl, 2-fluoroethyl, 2,2,2-trifluoroethyl, or pentafluoroethyl; hydroxy(C₁-C₆)alkyl can be alkyl substituted with 1 or 2 OH groups, such as hydroxymethyl, 1-hydroxyethyl, 2-hydroxyethyl, 1-hydroxypropyl, 2-hydroxypropyl, 3-hydroxypropyl, 1-hydroxybutyl, 4-hydroxybutyl, 3,4-dihydroxybutyl, 1-hydroxypentyl, 5-hydroxypentyl, 1-hydroxyhexyl, or 6-hydroxyhexyl; (C₁-C₆)alkoxycarbonyl can be methoxycarbonyl, ethoxycarbonyl, propoxycarbonyl, isopropoxycarbonyl, butoxycarbonyl, pentoxycarbonyl, or hexyloxycarbonyl; (C₁-C₆)alkylthio can be methylthio, ethylthio, propylthio, isopropylthio, butylthio, isobutylthio, pentylthio, or hexylthio; (C₂-C₆)alkanoyloxy can be acetoxy, propanoyloxy, butanoyloxy, isobutanoyloxy, pentanoyloxy, or hexanoyloxy; aryl can be phenyl, indenyl, indanyl, or naphthyl; and heteroaryl can be furyl, imidazolyl, triazolyl, triazinyl, oxazolyl, isoxazolyl, thiazolyl, isothiazolyl, pyrazolyl, pyrrolyl, pyrazinyl, tetrazolyl, pyridyl, (or its N-oxide), thienyl, pyrimidinyl (or its N-oxide), 1H-indolyl, isoquinolyl (or its N-oxide) or quinolyl (or its N-oxide).

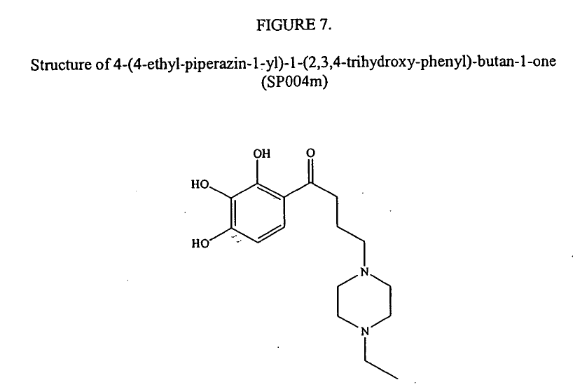

Compounds of formula I or II, including SP004, exhibit both AchE inhibition properties and σ-1 receptor agonist capacities. In one embodiment, SP004 generates an active metabolite, SP004m (FIG. 7), that also binds the σ-1 receptor and further exhibits antioxidant properties.

SP004's selectivity for human AchE (IC₅₀=1.3 μM) is 1.6-fold greater than that of galantamine and 6.8-fold greater than donezepil. Greig et al., Acta Neurol. Scand., 176: 74-84, 2000. SP004 selectively binds the σ-1 receptor (IC₅₀=680 nM, Ki=560 nM) over the σ-2 receptor (IC₅₀<10 μM). While not wishing to be bound by theory, SP004 may therefore lack the apoptotic and cytotoxic properties associated with σ-2 receptor agonists. Bowen, Pharm. Acta Helv., 74:211-218 (2000); Crawford et al., Eur. J. Pharmacol., 443:207-209 (2002).

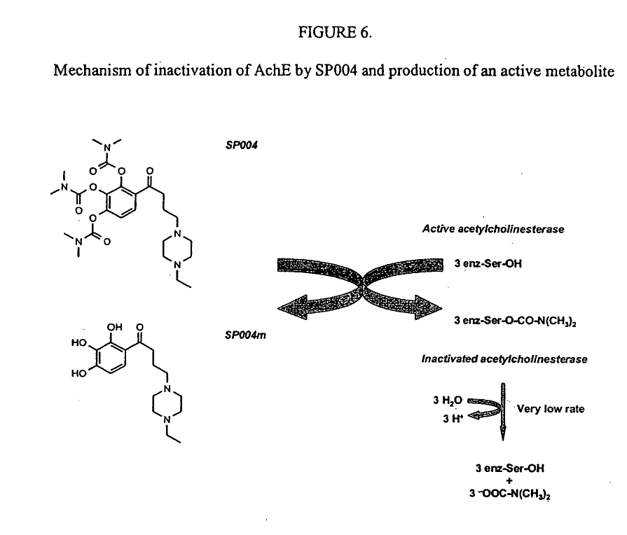

Upon or after inhibiting AchE, SP004 releases the active metabolite SP004m, as shown in FIG. 6. SP004 is a drug and a prodrug at the same time with the release of the active metabolite SP004m. While not wishing to be bound by theory, the structure of SP004m suggests that it is a σ-1 receptor ligand and has antioxidant properties. SP004m shares a common structure with SP004 and should therefore bind the σ-1 receptor and reinforce the activity of SP004. Moreover, the polyphenolic groups attached to SP004m after removal of the three carbamoyl groups from SP004 should confer strong antioxidant activity on SP004m, which would be beneficial to the oxidatively stressed AD brain. Plant polyphenols are known to have antioxidant properties, anticarcinogenic effects, anti-platelet properties, protective properties against cardiovascular disease, and neuroprotective properties against oxidative stress. Rice-Evans, Biochem. Soc. Symp., 61:103-116 (1995); Damianaki et al., J. Cell Biochem., 78:429-441 (2000); Soleas et al., Clin. Biochem., 35:119-124 (2002); Wang et al., Chin. Med. J., 115:378-380 (2002); Cuevas et al., Lipids, 35:143-148 (2000); Bastianetto et al., Br. J. Pharmacol., 131:711-720 (2000).

An effective amount of SP004 and/or SP004m, can be formulated with a pharmaceutically acceptable carrier to form a pharmaceutical composition before being administered for treatment of a disease or disorder. The pharmaceutical composition of the present invention that is administered to a subject should be administered in an amount that achieves a therapeutically effective dose of the compound of the invention in the blood serum of the subject, within or during a period of time, sufficient to elicit a desired therapeutic effect.

The amount of the therapeutic agent sufficient to elicit a therapeutic effect can be experimentally determined based on variables such as the absorption rate of the agent into the blood serum, the bioavailability of the agent, and the potency for treating the disorder. However specific dose levels of the therapeutic agents of the present invention for a particular subject depends upon a variety of factors including the activity of the specific compound used, the time of administration, the rate of excretion, the drug combination, the severity of the particular disorder being treated, the route of administration, and the age, body weight, general health, sex and diet of the subject, as is well understood by those skilled in the art.

The pharmaceutical compositions of the present invention can be administered by any appropriate route including, but not limited to, oral, nasogastric, rectal, transdermal, parenteral, subcutaneous, intramuscular, intravenous, intramedullary, intradermal, intranasal, transmucosal, vaginal topical, buccal, and sublingual. Such preparations may routinely contain buffering agents, preservatives, penetration enhancers, compatible carriers, and other therapeutic or non-therapeutic excipients as is well known to those skilled in the art.

Pharmaceutically acceptable cations include metallic ions and organic ions. Preferred metallic ions include, but are not limited to, appropriate alkali metal salts, alkaline earth meal salts, and other physiologically acceptable metal ions. Preferred organic ions include, but are not limited to, protonated tertiary amines and quarternary ammonium cations.

Pharmaceutically acceptable acids include, but are not limited to, hydrochloric acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid, oxalacetic acid, fumaric acid, propionic acid, aspartic acid, glutamic acid, benzoic acid, and the like.

Treatment dosages generally may be titrated to optimize safety and efficacy. Typically, dosage-effect relationships from in vitro or in vivo tests initially can provide useful guidance on the proper doses for subject administration. Studies in animal models generally may be used for guidance regarding effective dosages for treatment of gastrointestinal disorders or diseases in accordance with the present invention. In terms of treatment protocols, it should be appreciated that the dosage to be administered will depend on several factors, including the particular agent that is administered, the route administered, the condition of the particular subject, etc.

Generally speaking, one will desire to administer an amount of the compound that is effective to achieve a serum level commensurate with the concentrations found to be effective in vitro for a period of time effective to elicit a therapeutic effect. Thus, where a compound is found to demonstrate in vitro activity at, for example, a half-maximum effective dose of 200 nM, one will desire to administer an amount of the drug that is effective to provide about a half-maximum effective dose of 200 nM concentration in vivo for a period of time that elicits a desired therapeutic effect, for example, treating a disorder related to high beta-amyloid-induced neurotoxicity and other indicators as are selected as appropriate measures by those skilled in the art. Determination of these parameters is well within the skill of the art. These considerations are well known in the art and are described in standard textbooks. In order to measure and determine the effective amount of a compound of the present invention to be delivered to a subject, serum compound of the present invention concentrations can be measured using standard assay techniques.

The present compounds can also be used in combination with another pharmaceutical agent that is indicated for treating or preventing a neurodegenerative disease or disorder. When used in conjunction with the present invention, that is, in combination therapy, an additive or synergistic effect may be achieved such that many if not all of unwanted side effects can be reduced or eliminated. The reduced side effect profile of these drugs is generally attributed to, for example, the reduced dosage necessary to achieve a therapeutic effect with the administered combination.

The beneficial effect of combination therapy includes, but is not limited to, pharmacokinetic or pharmacodynamic co-action resulting from the combination of therapeutic agents. Administration of these therapeutic agents in combination typically is carried out over a defined time period, usually substantially simultaneously, minutes, hours, days, weeks, months or years depending upon the combination selected. Combination therapy generally is not intended to encompass the administration of two or more of these therapeutic agents as part of separate monotherapy regimens that incidentally and arbitrarily result in the combinations of the present invention.

Rather, combination therapy is intended to embrace administration of these therapeutic agents in a sequential manner, that is, where each therapeutic agent is administered at a different time, as well as administration of these therapeutic agents, or at least two of the therapeutic agents, in a substantially simultaneous manner. Substantially simultaneous administration can be accomplished, for example, in a combined dosage form or in separate dosage forms of the therapeutic compounds. Sequential administration of each therapeutic agent can be effected by any appropriate route. Combination therapy can also embrace the administration of the therapeutic agents as described above in further combination with other biologically active ingredients, such as, but not limited to, an analgesic, for example, and with non-drug therapies such as, but not limited to, surgery.

The therapeutic compounds of the combined therapy, whether administered simultaneously, substantially simultaneously, or sequentially, may involve a regimen calling for administration of one therapeutic compound by oral route and another therapeutic compound by an oral route, a percutaneous route, an intravenous route, an intramuscular route, or by direct absorption through mucous membrane tissues, for example. Whether the therapeutic compounds of the combined therapy are administered orally, by inhalation spray, rectally, topically, buccally, sublingually, or parenterally (for example, subcutaneous, intramuscular, intravenous and intradermal injections), separately or together, each such therapeutic compound will be contained in a suitable pharmaceutical formulation of pharmaceutically-acceptable excipients, diluents, or other formulation components.

For oral administration, the pharmaceutical composition can contain a desired amount of the compounds of the present invention, and be in the form of, for example, a tablet, a hard or soft capsule, a lozenge, a cachet, a troche, a dispensable powder, granules, a suspension, an elixir, a liquid, or any other form reasonably adapted for oral administration. Illustratively, such a pharmaceutical composition can be made in the form of a discrete dosage unit containing a predetermined amount of the active compound such as a tablet or a capsule. Such oral dosage forms can further comprise, for example, buffering agents. Tablets, pills, and the like additionally can be prepared with enteric coatings.

Pharmaceutical compositions suitable for buccal or sublingual administration include, for example, lozenges comprising the active compound in a flavored base, such as sucrose, and acacia or tragacanth, and pastilles comprising the active compound in an inert base such as gelatin and glycerin or sucrose and acacia. Liquid dosage forms for oral administration can include pharmaceutically acceptable emulsions, solutions, suspensions, syrups, and elixirs containing inert diluents commonly used in the art, such as water. Such compositions can also comprise, for example, wetting agents, emulsifying and suspending agents, and sweetening, flavoring, and perfuming agents.

The pharmaceutical compositions of the present invention can also be administered parenterally, as by injection (intravenous, intramuscular, subcutaneous). Such injectable compositions can employ, for example, saline, dextrose, or water as a suitable carrier material. The pH value of the composition can be adjusted, if necessary, with suitable acid, base, or buffer. Suitable bulking, dispersing, wetting, or suspending agents, including mannitol and polyethylene glycol (such as PEG 400), can also be included in the composition. A suitable parenteral composition can also include an active compound lyophilized in injection vials. Aqueous solutions can be added to dissolve the composition prior to injection.

The pharmaceutical compositions can be administered in the form of a suppository or the like. Carrier materials such as cocoa butter, theobroma oil, and other oil and polyethylene glycol suppository bases can be used in such compositions. Other carrier materials such as coatings (for example, hydroxypropyl methylcellulose film coating) and disintegrants (for example croscarmellose sodium and cross-linked povidone) can also be employed if desired.

The subject compounds may be free or entrapped in microcapsules, in colloidal drug delivery systems such as liposomes, microemulsions, and macroemulsions. All of the above pharmaceutical compositions can be prepared by any suitable method of pharmaceutics, which includes the step of bringing into association active compound of the present invention and a carrier material or carriers materials. In general, the compositions are uniformly and intimately admixing the active compound with a liquid or finely divided solid carrier, or both, and then, if necessary, shaping the product.

Tablets of the present invention can also be coated with a conventional coating material such as Opadry™ White YS-1-18027A (or another color) and the weight fraction of the coating can be about 3% of the total weight of the coated tablet. The compositions of the present invention can be formulated so as to provide quick, sustained or delayed release of the compositions after administration to the patient by employing procedures known in the art.

When the excipient serves as a diluent, it can be a solid, semi-solid, or liquid material which acts as a vehicle, carrier, or medium for the active ingredient. Thus, the compositions can be in the form of tablets, chewable tablets, pills, powders, lozenges, sachets, cachets, elixirs, suspensions, emulsions, solutions, syrups, aerosols (as a solid or in a liquid medium), soft and hard gelatin capsules, and sterile packaged powders.

In one embodiment of the present invention, the manufacturing processes may employ one or a combination of methods including: (1) dry mixing, (2) direct compression, (3) milling, (4) dry or non-aqueous granulation, (5) wet granulation, or (6) fusion. Lachman et al., The Theory and Practice of Industrial Pharmacy (1986).

Use of a long-term sustained release implant may be suitable for treatment of neurodegenerative diseases or disorders in patients who need continuous administration of the compositions of the present invention. “Long-term” release, as used herein, means that the implant is constructed and arranged to deliver therapeutic levels of the active ingredients for at least 30 days, and preferably 60 days. Long-term sustained release implants are well known to those of ordinary skill in the art and include some of the release systems described above.

Based on the description contained herein, one skilled in the art can utilize the present invention to its fullest extent. The following specific examples are therefore to be construed as merely illustrative and not limiting of the remainder of the disclosure in any way.

### EXAMPLE 1

Synthesis of SP004

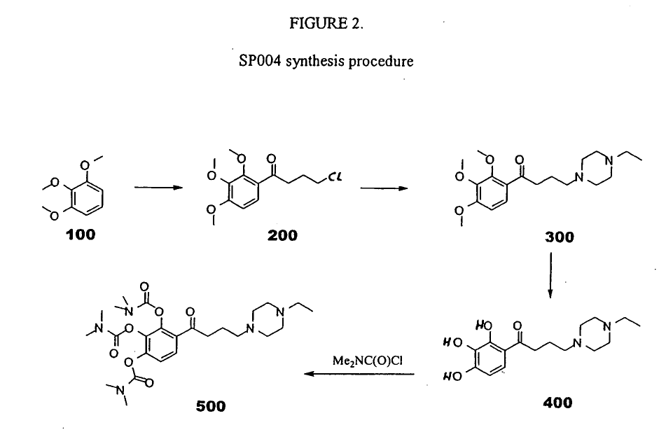

As depicted in FIG. 2, 10 grams (0.059 mol) 2,3,4-trimethoxy-phenyl (“100” in FIG. 2) was added to a suspension of aluminum chloride (35.5 g, 0.26 mol) in carbon disulfide. While the temperature was maintained at about 10° C., γ-chlorobutyryl chloride (14.7 g, 0.1 mol) was added. After the addition was completed, the stirring was continued for two hours at room temperature. The reaction mixture was poured onto ice and extracted with dichloromethane. The organic layer was separated, washed with water, and dried with MgSO₄. The solution was concentrated under reduced pressure. The residue was used in the next step without further purification.

In the next step, the compound produced above, 4-chloro-1-(2,3,4-trimethoxy-phenyl)-butan-1-one (“200” in FIG. 2) (7 g, 0.026 mol) and N-ethylpiperazine (5.8 g, 0.051 mol) were heated for seven hours at 100° C. After evaporation of the unreacted N-ethylpiperazine, the residue was chromatographed on silica gel.

In the next step, to a solution of the compound produced above, 4-(4-ethyl-piperazinyl)-1-(2,3,4-trimethoxy-phenyl)-butan-1-one (“300” in FIG. 2) (0.5 g, 0.0014 mol), in dry dichloromethane was added, under argon, borontribromide (1.7 g, 0.0071 mol). The solution was heated to reflux for 12 hours. Methanol was added, and the mixture was evaporated. After evaporation with methanol for several times, the residue was chromatographed on silica gel.

In the next step, a mixture of the compound produced above, 4-(4-ethyl-piperazin-1-yl)-1-(2,3,4-trihydroxy-phenyl)-butan-1-one (“400” in FIG. 2) (0.5 g, 0.0016 mol), dimethylcarbamoyl chloride (1.36 g, 0.0098 mol) and K₂CO₃ (1.05 g, 0.0098 mol) in dry acetonitrile was heated to reflux under argon for three hours. After dilution with water, the mixture was extracted with dichloromethane. The organic layer was dried over MgSO₄, the solvent was evaporated, and the residue was chromatographed on silica gel, to yield product SP004 (reference no. “500” in FIG. 2).

### EXAMPLE 2

SP004 Binding Assay

Different binding studies were performed with the following SP004 concentrations:

3E-10, 3E-9, 1E-8, 3E-8, 1E-7, 3E-7, 1E-6, 1E-5 M.

Materials and Methods

Central imidazoline-2 receptor (I₂). Central I₂ receptors extracted from rat cortex were used for this experiment. Increasing concentrations of SP004 were incubated for 30 minutes at 22° C. with 2 nM of the specific I₂ receptor ligand [³H]-idazoxan. Brown et al., Brit. J. Pharmacol., 99:803-809 (1990).

Muscarinic receptor (non-specific). Muscarinic receptors extracted from rat cortex were used for this experiment. Increasing concentrations of SP004 were incubated for 120 minutes at 22° C. with 0.05 nM of the muscarinic ligand [³H]-QNB. Richards, Brit. J. Pharmacol., 99:753-761(1990).

Neuronal nicotinic α-BGTX-insensitive receptor. Neuronal nicotinic α-BGTX-insensitive receptors extracted from rat cortex were used for this experiment. Increasing concentrations of SP004 were incubated for 75 minutes at 4° C. with 1.5 nM of the specific neuronal nicotinic α-BGTX-insensitive receptors ligand [³H]-cytisine. Pabreza et al., Mol. Pharmacol., 39:9-12 (1991).

Human recombinant sigma-1 receptor. Human recombinant sigma-1 receptors expressed in Jurkat cells were used for this experiment. Increasing concentrations of SP004 were incubated for 120 minutes at 22° C. with 8 nM of the sigma-1 receptor ligand [³H]-(+)-pentazocine. Ganaphthy et al., J. Pharmacol. Exp. Ther., 289:251-260 (1999).

Sigma-2 receptor. Sigma-2 receptors extracted from rat cortex were used for this experiment. Increasing concentrations of SP004 were incubated for 120 minutes at 22° C. with 5 nM of the sigma-2 receptor ligand [³H]-DTG. Bowen et al., Mol. Neuropharmacol., 3:117-126 (1993).

Results

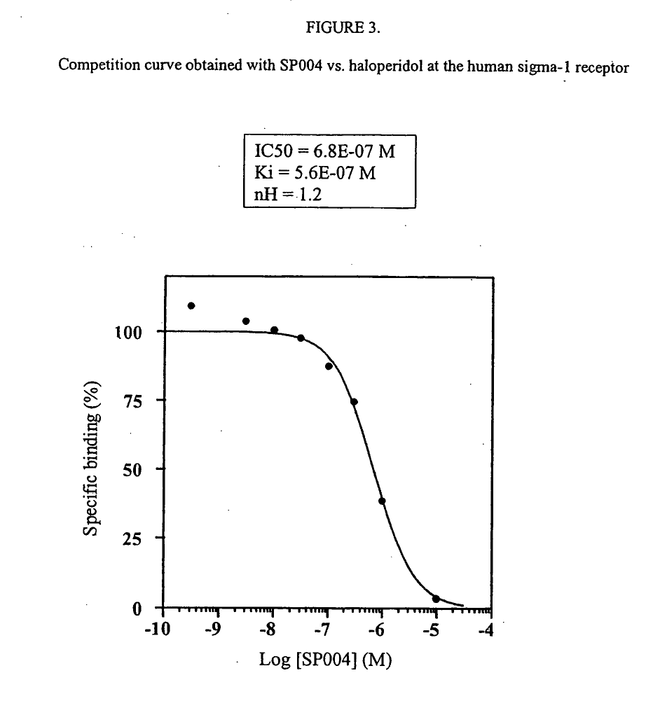

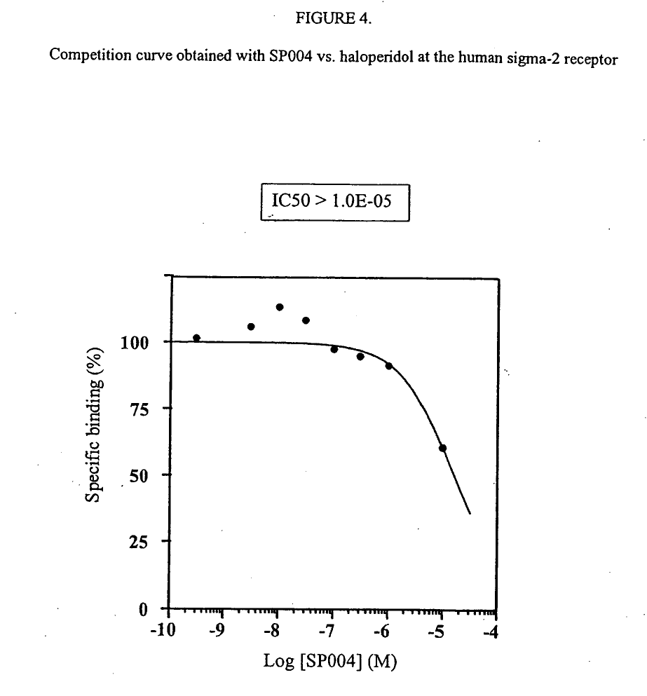

Results are summarized in FIG. 3, FIG. 4, and Table 1.

TABLE 1
Results of SP004 binding assays

| Receptor | IC₅₀(M) | Ki(M) | n_{H} |
|---|---|---|---|
| I₂ (central) | NC | — | — |
| M (non-specific) | NC | — | — |
| N (neuronal, α-BGTX | >1E−05 | — | — |
| insensitive) | | | |
| Sigma-1 (h) | 6.8E−07 | 5.6E−07 | 1.2 |
| Sigma-2 | >1E−05 | — | — |

Note:
IC₅₀ value is not calculable because of less than 25% inhibition at the highest tested concentration

Note: IC₅₀ value is not calculable because of less than 25% inhibition at the highest tested concentration.

As shown in Table 1, SP004 is not a ligand for the I₂, M, N (α-BGTX insensitive) and sigma-2 receptors. SP004 binds the sigma-1 receptor with IC₅₀=6.8E-07 M.

Acetylcholinesterase Assay

This assay was performed with the following SP004 concentrations: 3E-10, 1E-9, 3E-9, 1E-8, 3E-8, 1E-7, 3E-7, 1E-6, 3E-6, 1E-5 M.

Materials and Methods

Recombinant human acetylcholinesterase expressed in HEK-293 cells was used for this assay. AchE(h) was incubated for 30 minutes at 37° C. in the presence of the substrate AMTCh at 50 μM and with or without increasing concentrations of SP004. Ellman et al., Biochem. Pharmacol., 7:88-95 (1961).

Results

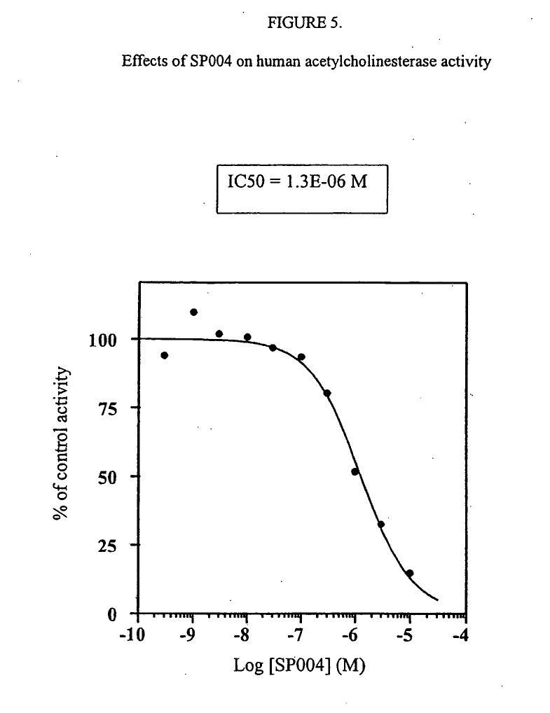

Results are shown in FIG. 5. As shown, SP004 inhibits AchE with an IC₅₀=1.3E-06 M and n_{H}=0.

Discussion

The data shows that the selectivity of SP004 for human AchE is IC₅₀ (1.3 μM), which is 1.6-fold more than galanthamine and 6.8-fold more than donezepil. Different reports have shown that the magnitude of the inhibition of AchE is not predictive of the magnitude of the increase of Ach concentration in the brain and does not correlate with the clinical benefit. Messamore et al., Neuropharmacology, 32:745-750 (1993); Isomae et al., Jpn. J. Pharmacol., (2002). Therefore, these preliminary results support the development of SP004 as a relevant AchEI. However, complementary studies like cerebral microdialysis are required to further confirm the data.

Even though these results seem very attractive, solely targeting AchE has been proven to slow down the degradation of mental status for only one to two years of treatment. As the modulation of the nicotinic receptor protects cells in cell culture against β-amyloid peptide, one way is to develop drugs that also target the nicotinic receptor, as does galanthamine. However, this strategy still targets the cholinergic pathway only.

In contrast, the present invention, while targeting the cholinergic pathway by inhibiting AchE, also targets the sigma-1 receptor. Consequently, the compound of the invention not only inhibits AchE, but through binding of the sigma-1 receptor at least also protects neuronal cells against cerebral ischemia, acts as an antidepressant, enhances acetylcholine release, and facilitates neurite sprouting. SP004 may also have anti-amnesic activity as a result of its activating effects on the cholinergic pathway.

In addition, SP004 also generates an active metabolite that can further enhance the effect of the novel compound by also binding the sigma-1 receptor and by acting as an antioxidant. As an antioxidant, SP004m may reduce the oxidative stress present in the neuronal cells affected by a neurodegenerative disease or disorder.

### EXAMPLE 3

Novel Sigma-1 Receptor Ligand with Acetylcholinesterase Inhibition Properties: A Nerve Agent Antidote

In modern war, protecting soldiers against any kind of threat and preserving their ability to fight has become a major concern of armies, as they have to face more and more deadly weapons on, and off, the battlefield. Nerve gas, like sarin, soman or Vx, is one of these deadly weapons. In addition to being deadly, nerve gas is easy to produce, can be made in large quantities and is easy to use. As such, nerve gas also constitutes an ideal weapon for terrorist organizations, as demonstrated by the attacks against the metro of Tokyo with sarin in the 1980's. Unprotected contact with nerve gas, so-called weapons of mass destruction, leads to certain death if appropriate treatment is not administered very quickly.

Sarin, soman and Vx all irreversibly inhibit the enzyme acetylcholinesterase (AchE), and as such, they all belong to the same family of neurotoxic gas. AchE degrades the neurotransmitter acetylcholine (Ach), regulating its concentration in the brain and therefore its activity. Inhibition of AchE induces an increase of Ach at the synaptic level and an over-activity of the brain cholinergic network. This results in seizures, cerebral hemorrhage, hypotension, cardiac failure and death. Because the binding of these neurotoxic agents to AchE is irreversible, the effects last until bound AchE is eliminated. At the neuronal level, the long lasting over-activation of the cholinergic network induces tremendous neuronal death, which leads to a high level of morbidity in an individual who survives such an exposure.

The “antidote” currently in use by the U.S. Army is an Ach antagonist, atropine. Atropine takes the place of Ach on the cholinergic receptor and blocks the activity of Ach. Unfortunately, permanent exposure to atropine is not desirable and the beneficial effect of atropine is transient, since the turnover of atropine is very fast compared to the long lasting activity of nerve gas.

As alternative treatment, SP004 may be a better antidote against nerve gas inhibition of AchE. SP004 was created as a multivalent chemical entity with affinity for various receptors and effect on various functions. SP004 acts as a reversible inhibitor of AchE (as demonstrated in vitro with human AchE (IC₅₀=1.3 μM). SP004 binds AchE at the same binding site as nerve gas, thus avoiding any lethal activity. Moreover, the “good timing” of the reversibility of the binding preserves the enzyme and renders it ready to function after the neurotoxic gas has been eliminated from the body. SP004 is a sigma-1 receptor ligand (human sigma-1 receptor IC₅₀=0.6 μM), and as such, preserves the mitochondria integrity and therefore, the energetic metabolism of the neuron. Also, the metabolite, which is also a sigma-1 receptor ligand, of SP004, possesses antioxidant properties contributing to counteract any oxidative stress induced by over-activation of the cholinergic system.

Thus, SP004 is unique in its ability to act on various neural receptor systems and at the same time increase the antioxidant defense of the body.

The invention has been described in an illustrative manner, and it is to be understood the terminology used is intended to be in the nature of description rather than of limitation. Obviously, many modifications, equivalents, and variations of the present invention are possible in light of the above teachings. Therefore, it is to be understood that within the scope of the appended claims, the invention may be practiced other than as specifically described.

All publications, patents and patent applications are incorporated herein by reference in their entirety.

What is claimed is:

1. A compound of formula (I): 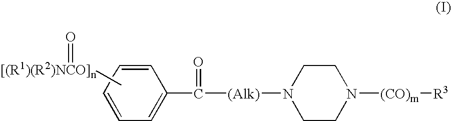
wherein
a) R¹ and R² are individually H, (C₁-C₆)alkyl, (C₃-C₆)cycloalkyl, (C₃-C₆)cycloalkyl((C₁-C₆)alkyl), (C₂-C₆)alkenyl, wherein cycloalkyl optionally comprises 1-2, S, nonperoxide O or N(R¹); aryl, aryl(C₁-C₆)alkyl, aryl(C₂-C₆)alkenyl, heteroaryl, heteroaryl(C₁-C₆)alkyl, or R¹ and R² together with the N to which they are attached form a 5- or 6-membered heterocyclic or heteroaryl ring, optionally substituted with R1 and optionally comprising 1-2, S, non-peroxide O or N(R¹);
b) (Alk) is (C₁-C₆)alkyl, (C₂-C₆)alkenyl, (C₃-C₆)cycloalkyl, (C₃-C₆)cycloalkyl(C₂-C₆)alkyl or [(C₂-C₆)alkyl(C₃-C₆)cycloalkyl[(C₃-C₆)alkyl]], each optionally substituted by 1-2 S, non-peroxide O or N(R¹);
c) n is 1, 2 or 3;
d) m is 0 or 1;
e) R³ is H, OH, (C₁-C₆)alkyl, (C₁-C₆)alkoxy, (C₃-C₆)cycloalkyl, (C₃-C₆)cycloalkoxy, (C₃-C₆)cycloalkyl((C₁-C₆)alkyl), (C₂-C₆)alkenyl, (C₂-C₆)alkynyl, (C₁-C₆)alkanoyl, halo(C₁-C₆)alkyl, hydroxyl(C₁-C₆)alkyl, (C₁-C₆)alkoxycarbonyl; (C₁-C₆)alkylthio, thio(C₁-C₆)alkyl- or (C₁-C₆)alkanoyloxy;
or a pharmaceutically acceptable salt thereof.

2. The compound of claim 1 wherein m is 0.

3. The compound of claim 1 wherein m is 1.

4. The compound of claim 1 wherein R³ is (C₁-C₆)alkyl, (C₃-C₆)cycloalkyl or (C₃-C₆)cycloalkyl(C₁-C₆)alkyl.

5. The compound of claim 4 wherein R³ is (C₁-C₄)alkyl.

6. The compound of claim 1 wherein (Alk) is (C₁-C₆)alkyl.

7. The compound of claim 6 wherein (Alk) is —(CH₂)₃—.

8. The compound of claim 1 wherein n is 3.

9. The compound of claim 8 wherein (R¹)(R²)NC(O)O is at the 2, 3 and 4 positions.

10. The compound of claim 9 wherein R¹ and R² are (C₁-C₄)alkyl.

11. The compound of claim 1 wherein R¹ and R² are methyl, m is 0 and R³ is ethyl.

12. The compound of claim 1, which is a compound of formula (II):

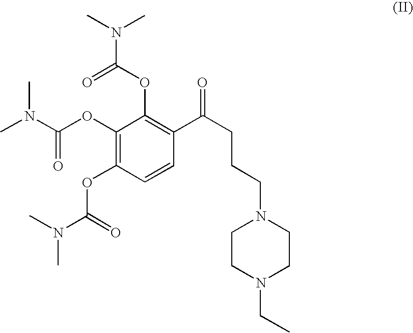

13. The compound of claim 1, which is 4-(4-ethyl-piperazin-1-yl)-1-(2,3,4-trihydroxy-phenyl)-butan-1-one.

14. A method of treating a mammal threatened or afflicted by a neuropathological condition comprising administering an effective amount of a compound of formula I: 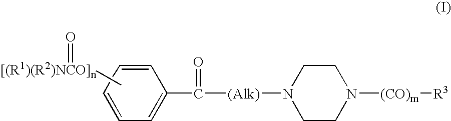
a) R¹ and R² are individually H, (C₁-C₆)alkyl, (C₃-C₆)cycloalkyl, (C₃-C₆)cycloalkyl((C₁-C₆)alkyl), (C₂-C₆)alkenyl, wherein cycloalkyl optionally comprises 1-2, S, nonperoxide O or N(R¹); aryl, aryl(C₁-C₆)alkyl, aryl(C₂-C₆)alkenyl, heteroaryl, heteroaryl(C₁-C₆)alkyl, or R¹ and R² together with the N to which they are attached form a 5- or 6-membered heterocyclic or heteroaryl ring, optionally substituted with R1 and optionally comprising 1-2, S, non-peroxide O or N(R¹);
b) (Alk) is (C₁-C₆)alkyl, (C₂-C₆)alkenyl, (C₃-C₆)cycloalkyl, (C₃-C₆)cycloalkyl(C₂-C₆)alkyl or [(C₂-C₆)alkyl(C₃-C₆)cycloalkyl[(C₃-C₆)alkyl], each optionally substituted by 1-2 S, non-peroxide O or N(R¹);
c) n is 1, 2 or 3;
d) m is 0 or 1;
e) R³ is H, OH, (C₁-C₆)alkyl, (C₁-C₆)alkoxy, (C₃-C₆)cycloalkyl, (C₃-C₆)cycloalkoxy, (C₃-C₆)cycloalkyl((C₁-C₆)alkyl), (C₂-C₆)alkenyl, (C₂-C₆)alkynyl, (C₁-C₆)alkanoyl, halo(C₁-C₆)alkyl, hydroxyl(C₁-C₆)alkyl, (C₁-C₆)alkoxycarbonyl; (C₁-C₆)alkylthio, thio(C₁-C₆)alkyl- or (C₁-C₆)alkanoyloxy;
or a pharmaceutically-acceptable salt thereof in combination with a carrier or excipient.

15. The method of claim 14, wherein the neuropathological condition is the result of a stroke, heart attack or exposure to a neurotoxic agent.

16. The method of claim 14, wherein the neuropathological condition is Alzheimer's disease, Parkinson's disease, Huntington's disease, Amyotrophic Lateral Sclerosis, Down's Syndrome or Korsakoff's disease.

17. A method of treating a neurodegenerative disease or disorder in a subject comprising administering to the subject a compound of formula (I):

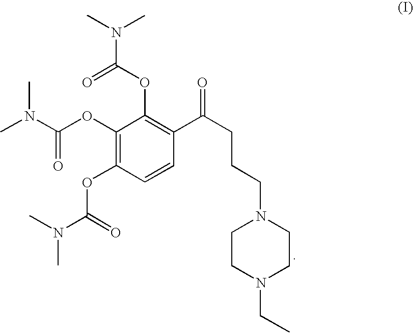

18. The method of claim 17, wherein the neurodegenerative disease or disorder is the result of a stroke, heart attack or exposure to a neurotoxic agent.

19. The method of claim 17, wherein the neurodegenerative disease or disorder is Alzheimer's disease, Parkinson's disease, Huntington's disease, Amyotrophic Lateral Sclerosis, Down's Syndrome or Korsakoff's disease.

20. A pharmaceutical composition comprising a therapeutically effective amount of a compound of claim 1.

21. The pharmaceutical composition of claim 20, further comprising at least one pharmaceutically acceptable excipient.

22. The pharmaceutical composition of claim 21, wherein the pharmaceutically acceptable excipient comprises a binder, a disintegrant, a filler, a surfactant, a solubilizer, a stabilizer, a lubricant, a wetting agent, a diluent, an anti-adherent, a glidant, or a pharmaceutically compatible carrier.

23. The pharmaceutical composition of claim 20, wherein the compound is in a dosage form selected from the group consisting of tablet, soft gelatin capsule, hard gelatin capsule, suspension tablet, effervescent tablet, powder, effervescent powder, chewable tablet, solution, suspension, emulsion, cream, gel, patch, and suppository.

24. The pharmaceutical composition of claim 20, wherein the compound generates a metabolite that is useful for treating neurodegenerative diseases or disorders.
